(19) United States
(12) Patent Application Publication
(10) Pub. No.: US 2026/0130658 A1
(43) Pub. Date: May 14, 2026

(54) MINIMALLY INVASIVE DEVICE FOR NERVE IDENTIFICATION, STIMULATION, AND MANIPULATION

(71) Applicant: The General Hospital Corporation, Boston, MA (US)

(72) Inventors: Lisa Gfrerer, Boston, MA (US); Shriya S. Srinivasan, Boston, MA (US); William G. Austen, Jr., Boston, MA (US)

(21) Appl. No.: 19/118,700

(22) PCT Filed: Oct. 9, 2023
(86) PCT No.: PCT/US2023/076364

§ 371 Date: Apr. 4, 2025

Related U.S. Application Data
(60) Provisional application No. 63/378,850, filed on Oct. 7, 2022.

(51) Int. Cl. A61B 17/00

(52) U.S. Cl. CPC A61B 17/00234; A61B 2017/00026; A61B 2017/00238

(57) ABSTRACT

Systems and methods for an insertable medical device are described. The device comprises a device body configured to be insertable into a subject; proximal and distal openings; a cavity extending through the device body and in communication with the proximal opening and the distal opening, the cavity and the proximal opening being dimensioned to receive an instrument therein; a plurality of electrically conductive pads configured to be positioned a predetermined location relative to the proximal and distal openings, and configured to sense an electrical characteristic of the subject; an I/O device configured to: output signal data from the electrically conductive pads to a processing device operating a ML algorithm, the signal data including data corresponding to the electrical characteristic of the subject, and receive response data generated by the ML algorithm indicating a tissue type proximate respective electrically conductive pads based on an analysis of the signal data.

### CROSS-REFERENCE TO RELATED APPLICATIONS

This application claims the benefit of U.S. Provisional Patent Application No. 63/378,850, filed Oct. 7, 2022, the entire contents of which are herein incorporated by reference for all purposes.

### TECHNICAL FIELD

The present disclosure relates to minimally invasive systems and methods for identifying structures (e.g., nerve tissue) beneath the skin surface and other tissues and for systems and methods for performing procedures on the tissue using such information.

### BACKGROUND

Surgical transection of peripheral nerves (neurectomies) is performed for different disease processes. Neurectomies of motor nerves permanently paralyze muscles and have areas of application such as improvement of spasticity after spinal cord injury and stroke, as well as improvement of involuntary muscle spasms. In addition, neurectomies can be performed for denervation of muscles to prevent nerve compression that can occur in the glabellar muscle group and cause migraine. Some surgical approaches are using motor nerve neurectomies for prevention of facial wrinkles, back pain and spasm, cervical dystonia, overactive bladder, and Raynaud's disease. Further, neurectomies of sensory nerves can be performed to prevent chronic pain.

In comparative examples, neurectomies of both motor and sensory nerves are performed in an open surgical fashion. For motor nerves, surgeons open the anatomic area of interest and use nerve stimulators to identify the motor nerve branches that will be transected. Then, using either scissors or a scalpel blade, the nerve is divided.

For sensory nerves, there is no stimulation device, so the surgeon must rely on anatomic knowledge of the nerve location. Once identified, the nerve is divided with scissors, cautery or a blade. Currently, there is no minimally invasive device that is able to stimulate motor and sensory nerves and perform neurectomies without making an open incision. There exists a need for a minimally invasive technology to perform nerve stimulation, neurectomies, and other procedures via different mechanisms.

### SUMMARY

The present disclosure provides for minimally invasive devices and methods of manufacturing and operating such a device. In addition to the cases described above, the devices and methods described herein may be used for cases including sensory nerve transection; motor or mixed nerve transection; sensory organ denervation by nerve transection; muscle denervation by innervating nerve transection; injection of fluids or solids into nerves; evaluation of nerve health, conduction, viability, hydration status, and color before, after, and/or during surgery; evaluation of nerve health, mapping of nerves for dissection and/or research; proxy as a surgical planning tool; freezing, burning, cutting, and/or segmentally cutting a nerve or other tissue; coagulation of tissues; acid treatment of tissues; magnetically cutting tissues; crushing neural or other structures; inducing an avulsion; nerve specific energy blading; laser treatments; cutting and/or capping a nerve; placement of catheters; injection of vessels near nerves; deposition of drugs and/or hardware into tissue in a minimally invasive manner targeting or avoiding a nerve; or combinations thereof.

The systems and methods described herein may serve several conditions, including wrinkles; migraine; bladder spasm; incontinence; pain ablation; back pain; anesthesia (e.g. for craniotomies); epidurals; neuroma ablation, treatment, and/or identification; abdominal hernia; plexus blocks; prevention of metastasis; abdominal wall relaxation; synkinesis; occipital neuralgia; muscle spasm; chronic pain; acute pain; or combinations thereof.

In accordance with one aspect of the present disclosure, an insertable medical device is provided. The insertable medical device comprises a device body extending from a proximal end to a distal end and configured to be insertable into a subject; a proximal opening at the proximal end of the device body; a distal opening at or near the distal end of the device body; a cavity extending through the device body and in communication with the proximal opening and the distal opening, the cavity and the proximal opening being dimensioned to receive an instrument therein; a plurality of electrically conductive pads configured to be positioned a first predetermined distance from the proximal opening and a second predetermined distance from the distal opening, and configured to sense an electrical characteristic of internal tissue of the subject when the distal end is inserted into the subject; and an input/output (I/O) device configured to: output signal data from the plurality of electrically conductive pads to a processing device, the signal data including data corresponding to the electrical characteristic of the subject, and receive response data differentiating a tissue type proximate respective ones of the plurality of electrically conductive pads based on an analysis of the signal data.

In accordance with another aspect of the present disclosure, a method of differentiating tissue is provided. The method comprises receiving signal data from a plurality of electrically conductive pads located a first predetermined distance from a proximal opening in a device body and located a second predetermined distance from a distal opening in the device body, the plurality of electrically conductive pads being located under a surface of a skin of a subject; analyzing the signal data to distinguish electrical characteristics of different structures internal to the subject; and generating an output that indicates a target tissue is proximate respective ones of the plurality of electrically conductive pads based on analysis of the signal data.

In accordance with another aspect of the present disclosure, a method of identifying a tissue is provided. The method comprises receiving, from an insertable medical device inserted in a subject tissue, a measurement value of an electrical and/or optical characteristic; applying a machine learning (ML) model to the measurement value; determining, by the ML model, a tissue type based on the measurement value; and outputting an indication of the tissue type.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure.

FIG. 1 illustrates an example of a minimally invasive system in accordance with various aspects of the present disclosure.

FIGS. 2A-2C respectively illustrate examples of an insertable medical device in accordance with various aspects of the present disclosure.

FIG. 3 illustrates an example of a processing device in accordance with various aspects of the present disclosure.

FIG. 4 illustrates an example of a method of identifying a tissue type in accordance with various aspects of the present disclosure.

FIG. 5A illustrates an example of an insertable medical device inserted into a subject in accordance with various aspects of the present disclosure.

FIG. 5B illustrates an example of measurement data in accordance with various aspects of the present disclosure.

FIGS. 5C-5D respectively illustrate examples of a feedback interface for the example situation of FIG. 5A.

FIGS. 6A-6M respectively illustrate examples of a method of operating a medical device in accordance with various aspects of the present disclosure.

FIG. 7 illustrates an example of a process flow for operating a medical device in accordance with various aspects of the present disclosure.

In the drawings, unless otherwise indicated, the same reference numerals and characters are used to denote like features, elements, components, or portions of the illustrated examples.

### DETAILED DESCRIPTION

Before the present invention is explained in greater detail, it is to be understood that the invention is not limited in its application to the details of construction and the arrangement of components expressly set forth in the following description or illustrated in the associated drawings. The invention is capable of other embodiments or examples and of being practiced or carried out in various ways.

As used herein, the singular forms “a,” “and,” and “the” include plural forms unless the context clearly indicates otherwise. The use of the terms “including,” “comprising,” or “having” and variations thereof is meant to encompass the items listed thereafter and equivalents thereof as well as additional items. Examples referenced as “comprising”, “including”, or “having” certain elements are also contemplated as including corresponding examples “consisting essentially of” and “consisting of” those elements, unless the context clearly dictates otherwise. Where the phrasing “at least one of A, B, or C” is used, it is intended to denote A, or B, or C, or combinations thereof (e.g., A and B; A and C; B and C; or A, B, and C). Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10. Further, as used herein, ranges that are between two particular values should be understood to expressly include those two particular values. For example, “between 0 and 1” means “from 0 to 1” and expressly includes 0 and 1 and anything falling inside these values. Also, as used herein “about” means ±20% of the stated value, and includes more specifically values of ±10%, ±5%, ±2%, ±1%, and ±0.5% of the stated value.

As used herein, unless otherwise defined or limited, directional terms are used for convenience of reference for discussion of particular figures or examples. For example, references to upper, lower, upward, downward, or other directions may be used to discuss aspects of a particular example or figure, but do not necessarily require similar orientation or geometry in all installations or configurations.

The following discussion is presented to enable a person skill in the art to make and use embodiments of the invention. Various modifications to the illustrated examples will be readily apparent to those skilled in the art, and certain principles herein can be applied to other examples and applications without departing from the scope of the invention. Thus, the present invention is not intended to be limited to examples expressly shown, but are to be accorded the widest scope consistent with the principles and features disclosed herein. The following detailed description is to be read with reference to the figures, in which like elements in different figures have like reference numerals. The figures, which are not necessarily to scale, depict selected examples and are not intended to limit the scope of embodiments of the invention. Skilled artisans will recognize that the examples provided herein have many useful alternatives which fall within the scope of the present disclosure.

As noted above, comparative systems and methods for detecting and performing procedures on certain tissue types, such as nerves, require an open surgical margin and/or a high degree of anatomic knowledge of tissue locations. Moreover, comparative systems and methods may be incapable of differentiating tissue types at small scales. The systems and methods set forth herein enable the minimally invasive identification and visualization of tissue types even at millimeter (mm) or sub-mm scales, and permit the performance of procedures such as tissue stimulation and tissue manipulation in a similarly minimally invasive manner.

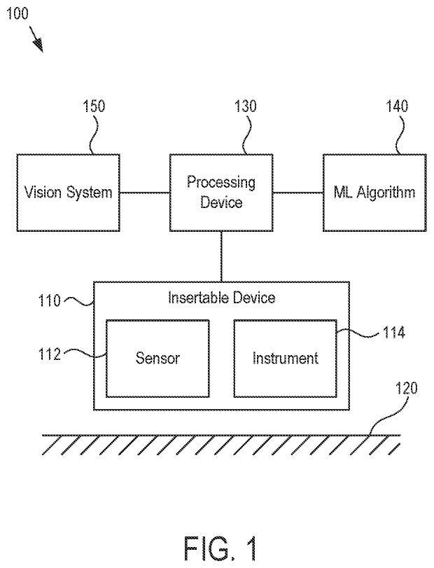

FIG. 1 illustrates a system 100 for resolving the above-described needs and achieving the above-described benefits. The system 100 includes an insertable medical device 110 which includes and/or is configured to receive therein a sensor device 112 and an instrument 114. The sensor device 112 identifies, detects, and/or differentiates between tissue types in a subject tissue 120, and may be referred to in some implementations as a detection tool. The tissue type may be at least one of a muscle tissue, a nerve tissue, a fat tissue, a bone tissue, a blood vessel tissue, a tumor tissue, and the like. The instrument 114 is used to perform certain procedures on the subject tissue 120. Both the sensor device 112 and the instrument 114 are described in more detail below. In the illustrated example, the insertable device 110 is operatively connected to a processing device 130, which is in turn operatively connected to a machine learning (ML) model 140 and a vision system 150. However, in other examples, the ML model 140 and/or the vision system 150 may be connected to one another and/or directly connected to the insertable device 110 and/or its components.

While FIG. 1 illustrates the insertable device 110, the processing device 130, the ML model 140, and the vision system 150 as separate entities, the present disclosure is not so limited. In some implementations, the processing device 130 and/or the vision system 150 may be a component of the insertable device (or vice versa), the ML model 140 may reside in the processing device 130 (e.g., in a memory thereof), the vision system 150 and the processing device 130 may be integrated into a single device, and so on. In short, the insertable device 110, the processing device 130, the ML model 140, and the vision system 150 may be integrated and/or distributed in any combination among one or a number of separate devices or housings. Moreover, where any one or more of the insertable device 110, the processing device 130, the ML model 140, and the vision system 150 are separate from one another, a connection between separate entities may be a physical connection (e.g., a copper wire, an Ethernet cable, an optical fiber, etc.) and/or a remote connection (e.g., a Wi-Fi connection, a Bluetooth connection, a near field communication (NFC) connection, a free-space optical connection, etc.). In some implementations, the processing device 130 and/or the ML model 140 may be cloud-based.

In one particular example, the insertable device 110 is configured to provide access to a subcutaneous space, such as a fascial and/or muscular space, for the sensor 112 and/or the instrument 114. In implementations, the insertable device 110 may include an incising tip, or may receive an incising tip associated with the instrument 114 therethrough. The incising tip is sufficiently sharp to penetrate the skin of the subject tissue 120, for example to make a small incision in the skin of about 1 centimeter (cm) or smaller. The incising tip may also have slitted edges to allow blunt dissection into subcutaneous tissues and fascial layers. The incising tip may be a needle and may include a port to insert an endoscope. In some implementations, ultrasound may be used in combination with the insertable device to enable visualization.

The sensor device 112 may be a nerve detection probe with either a unipolar or bipolar lead (e.g., formed of stainless steel, platinum, iridium, or copper) and may have an impedance of less than 10 kiloohm (kΩ). Through the insertable device (e.g., via a cavity formed therein), the nerve detection probe may be advanced into the subcutaneous space, fascial, and muscular space. The sensor device 112 may include or communicate with an impedance analyzer attached to the nerve detection probe, which may continuously or continually scan the subject tissue 120 at 50-1000 Hertz (Hz). In some implementations, the impedance analyzer may be instead included in the processing device 130. In either implementation, upon detection of a significant change in an electrical characteristic (e.g., an impedance drop) of the subject tissue 120, the impedance analyzer may indicate the presence of a particular type of tissue, such as neural tissue. Upon contact with tissue, the impedance analyzer or another detection meter may output pulses.

Square, trapezoidal, and triangle wave pulses having amplitudes of 0.5-10 milliamps (mA), frequencies of 1-80 Hz, trains of 2-55 pulses, and pulse widths of 5-200 microseconds (μs) may enable the detection of fibers corresponding to smooth and skeletal muscle targets. Motor nerve activity can be detected using visual confirmation of muscle contraction, palpation of muscle twitch or tetanic contraction, surface electromyography (EMG) measurements, intramuscular needle EMG measurements, cuff electrodes in other areas of the body, implants (such as DBS probes), nerve conduction velocity on a bipolar probe, or combinations thereof. Sensory nerve activity can be detected using patient reported sensations, sensory evoked potentials on the innervated area, nerve conduction velocity on a bipolar probe, or combinations thereof.

In this example, the instrument 114 may be a hook probe to be inserted via the insertable device 110 upon target identification. The hook probe may consist of one to five prongs that enable firm attachment of the insertable device 110 to the subject tissue 120. Another tool may be inserted alongside the hook probe. For example, scissors may be inserted to enable further dissection of the tissue for target isolation. Additionally or alternatively, cautery, cooling or heating agents, radiofrequency (RF) devices, scissors, or a press-fit blade shaped to the contours of the hook probe may be used to transect the target nerve. The instrument 114 may be or include a tool to perform cryotherapy through the cavity of the insertable device 110, thereby to temporarily freeze the nerve. Following evaluation of the functional outcomes, a second procedure may be performed to refreeze the nerve, ablate it, or transect it. The use of heat to damage or temporarily disable the nerve may also be used. To achieve this, the instrument 114 may be capable of creating temperatures up to 200° C. via the cavity of the insertable device 110 and used to perform thermo-ablation of the nerve. The instrument 114 may additionally or alternatively have the ability to cap the nerve end.

The instrument 114 may retain the capacity to locally deliver drugs to the site of transection, for example to minimize post-operative pain or irregular sensations. The instrument 114 can also be used to deliver anesthetic agents and/or botulinum toxin. Chemical or pharmacological agents may be delivered to the site of the subject tissue 120 for targeted therapy. These drugs may include neurotoxins, anesthetic agents, regenerative agents, immunotherapies, and/or cell-based therapies. Viral vectors may also be delivered using the instrument 114. This may, for example, be used to deliver the genetic material for transfecting opsin channels that are light activated for optogenetic applications such as nerve stimulation or activation control. Dyes can also be delivered, which will travel in anterograde or retrograde directions along the axons. These can be used to trace neural anatomy for surgical planning, intraoperative dissection, or resection. Additionally, dyes can inform an operator of the system 100 on the health, location, vascularization, or status of a nerve.

After transection, the nerve detection probe or tool inside its cannula may be reinserted to provide visual and/or electrophysiological confirmation of transection. In some implementations, an optical fiber may be included in the insertable device 110 alongside an LED-based light source to enable visualization of the subject tissue 120 and clarify dissections.

In some examples the insertable device 110 and the sensor device 112 may be unitary in construction, whereas in other examples the insertable device 110 and the sensor device 112 may be separate (e.g., with the insertable device 110 dimensioned so as to receive the sensor device 112 therein). In other words, the sensor device 112 may be the implantable medical device itself, or the sensor device 112 may be contained within a sheath corresponding to the implantable medical device.

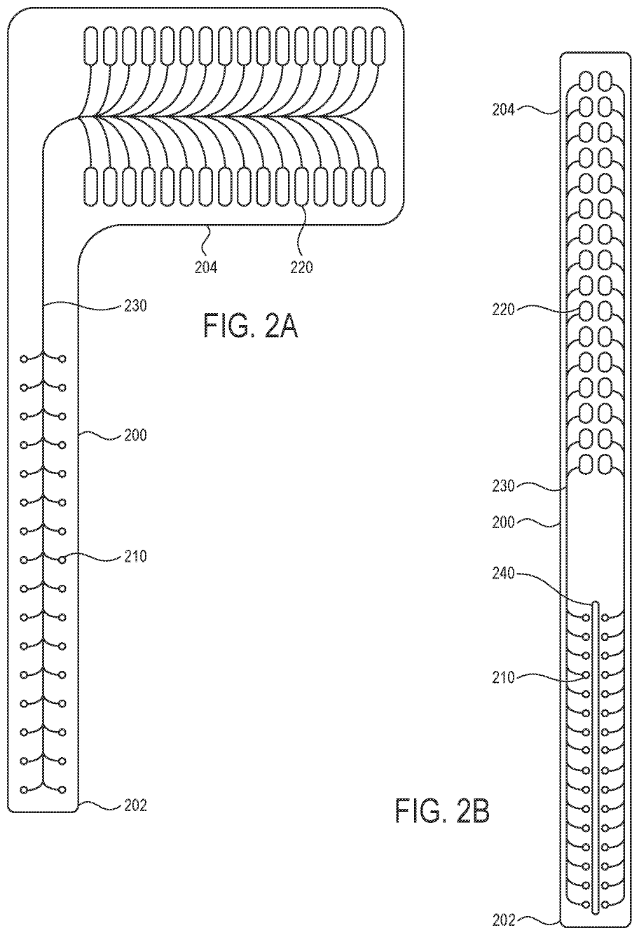

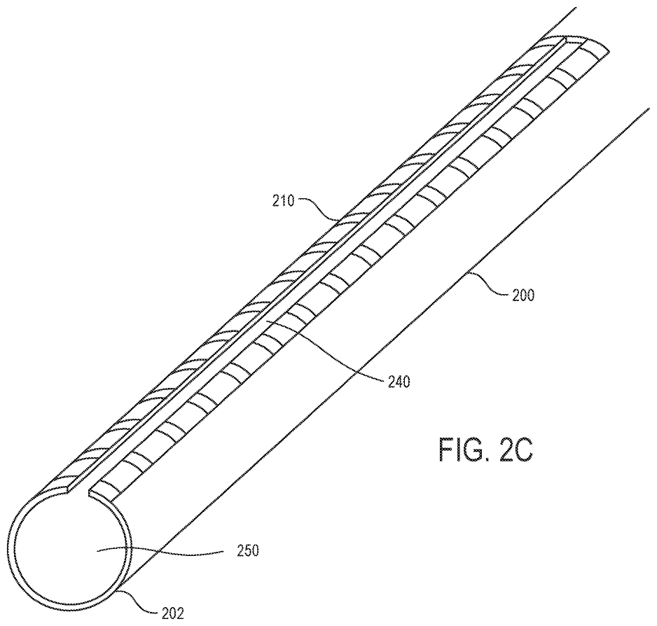

The sensor device 112 may have a device body that is elongated (e.g., a needle-like body). In some examples, the device body may have a fixed sharp tip (e.g., a cutting tip, a needle-like insertion tip, etc.), a blunt tip (e.g., a blunt dissection tip), or interchangeable tips. The device body may be rigid, flexible, or a combination thereof; and may be straight, curved, or a combination thereof. Three examples of the sensor device 112 are illustrated in FIGS. 2A-2C.

In each of the examples of FIGS. 2A-2C, the sensor device 112 includes a sensor body 200 configured to be insertable into a subject (e.g., into the subject tissue 120). In implementations where the sensor device 112 and the insertable device 110 are unitarily constructed, the sensor body 200 may include a proximal opening at a proximal end 204 of the sensor body 200 and a distal opening at or near a distal end 202 of the sensor body 200, with a cavity extending through the sensor body 200 and in communication with the proximal opening and the distal opening. In such implementations, the cavity and the proximal opening may be dimensioned to receive an instrument (e.g., the instrument 114) therethrough. In implementations where the sensor device 112 and the insertable device 110 are separate, the insertable device 110 may instead include the proximal opening, the distal opening, and the cavity; alternatively, both the insertable device 110 and the sensor device 112 may include respective proximal openings, distal openings, and cavities. The cavity and the proximal opening may be dimensioned to receive an instrument (e.g., the instrument 114) and/or a detection tool (e.g., the sensor device 112) therethrough. In either implementation, the cavity may instead be multiple cavities; for example, the insertable device 110 may have a first cavity to receive the instrument 114 and a second cavity to receive the sensor device 112. The distal opening may be parallel and/or normal to the sensor body 200. A parallel distal opening may be used to expose an inserted sharp or blunt tip (e.g., corresponding to an instrument inserted through the proximal opening). The cavity may be symmetric or asymmetric.

In each of the examples of FIGS. 2A-2C, the sensor device 112 includes a plurality of electrically conductive pads 210, each of which are configured to be positioned a first known (i.e., predetermined) distance from the proximal opening and a second known (i.e., predetermined) distance from the distal opening. The plurality of electrically conductive pads 210 are configured to sense an electrical characteristic and/or to apply an electrical signal to a portion of the subject tissue 120 proximate the plurality of electrically conductive pads 210. The electrical characteristic may be a “local” impedance in the area of subject tissue 120 in contact with a respective electrically conductive pad 210, measured with respect to another area of the subject tissue 120 in contact with another electrically conductive pad 210 or with respect to a common ground. The electrical signal may be a voltage and/or a current. In some implementations, the plurality of electrically conductive pads 210 may be configured to stimulate the subject tissue 120 with the electrical signal. In such implementations, the insertable device 100 may be capable of sensing and treating simultaneously with a single tool.

In the illustrations of FIGS. 2A-2C, the plurality of electrically conductive pads 210 are arranged in a 16×2 array, with 32 electrically conductive pads 210 in total; however, the present disclosure is not limited to 32 total electrically conductive pads 210, is not limited to a two-column array, and is not limited to a regular array. Moreover, while FIGS. 2A-2C illustrate examples in which all of the electrically conductive pads 210 are the same size, in other implementations the electrically conductive pads 210 may have a variety of sizes, including sizes different from one another. The plurality of electrically conductive pads 210 may be dimensioned so as to ensure that each pad 210 does not touch multiple different tissues of the same target tissue type (e.g., two different nerves) simultaneously, while also ensuring that each pad 210 is large enough to have a sufficient contact area with the tissue. Each electrically conductive pad 210 may be identified by an index number. For example, the upper-left electrically conductive pad 210 in FIG. 2A may be the “first electrically conductive pad” or “electrically conductive pad 1,” the lower-left electrically conductive pad 210 in FIG. 2A may be the “sixteenth electrically conductive pad” or “electrically conductive pad 16,” the upper-right electrically conductive pad 210 in FIG. 2A may be the “seventeenth electrically conductive pad” or “electrically conductive pad 17,” the lower-right electrically conductive pad 210 in FIG. 2A may be the “thirty-second electrically conductive pad” or “electrically conductive pad 32,” and so on.

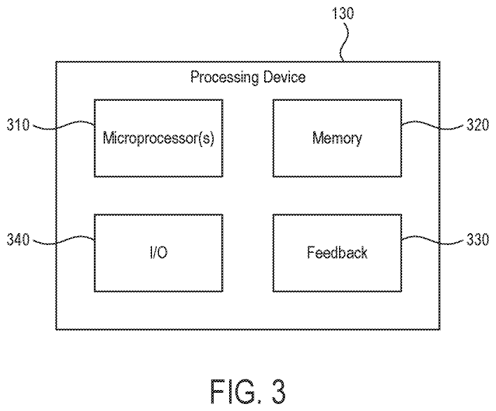

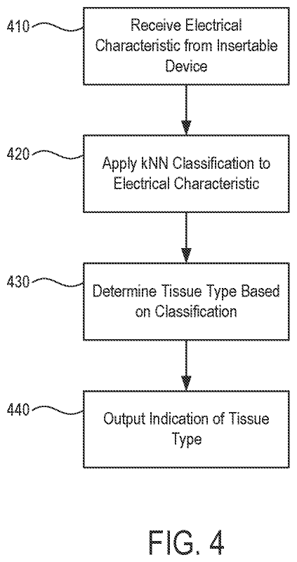

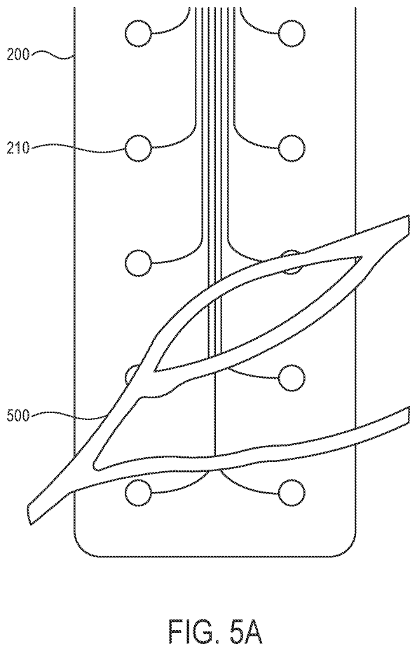

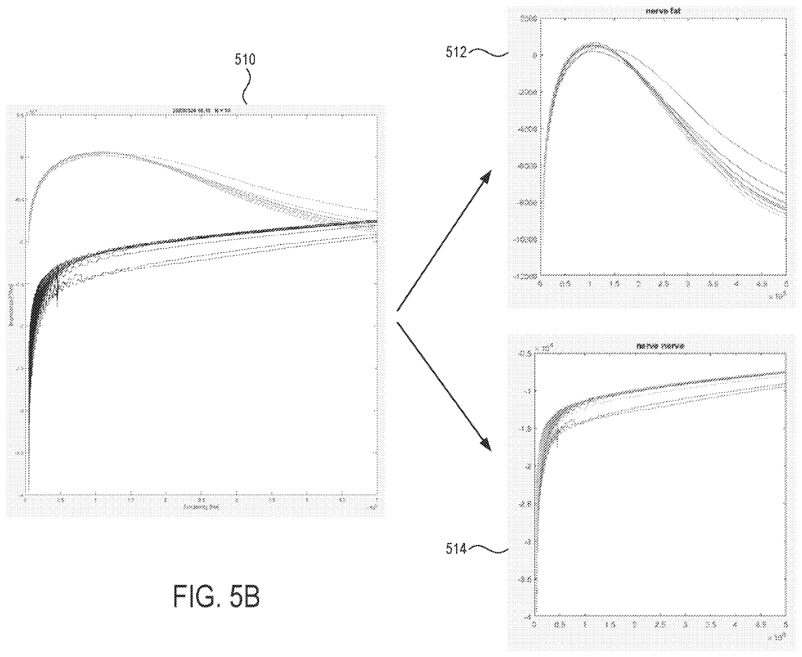

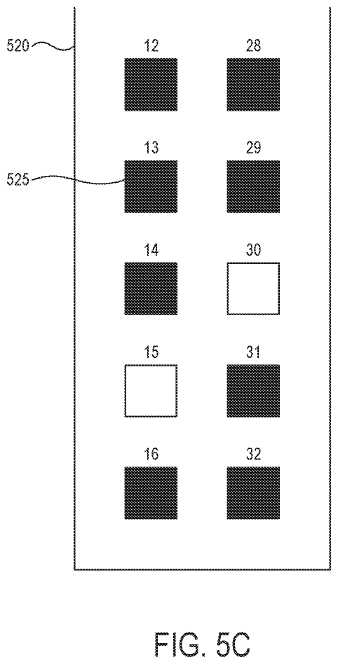

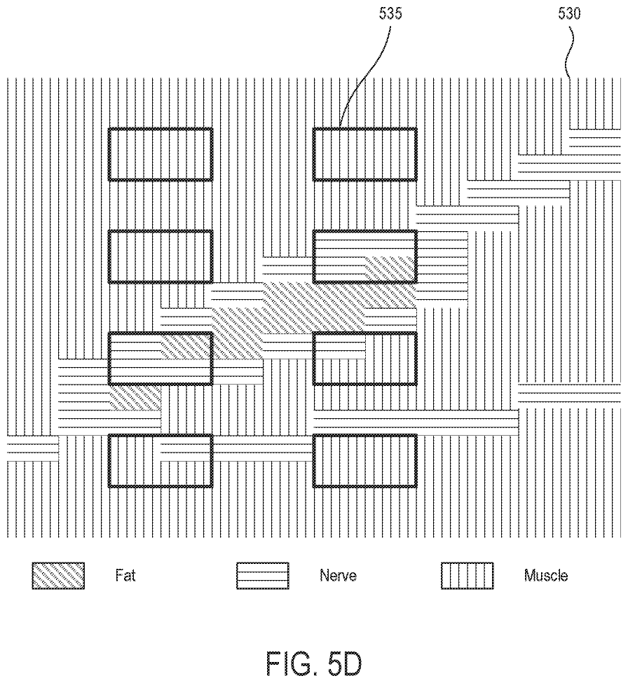

In each of the examples of FIGS. 2A-2C, the plurality of electrically conductive pads 210 are connected to a plurality of external pads 220 by a set of conductive paths 230 in a one-to-one manner, such that a first electrically conductive pad 210 is connected to a first external pad 220, a second electrically conductive pad 210 is connected to a second external pad, and so on. Each of the external pads 220 may be identified by the same index number as the electrically conductive pad 210 to which the external pad 220 is connected. Generally, the plurality of conductive paths 230 will be separate from one another so as to avoid cross-talk, as can be seen in the more detailed view of FIG. 3 as discussed in more detail below. The plurality of conductive paths 230 may be disposed on or near an exterior surface of the sensor body 220, as shown in FIGS. 2A and 2B, or may be disposed within the sensor body 220 and/or at or near an interior surface of the sensor body 220, as may be the case with FIG. 2C. The plurality of external pads 220 are configured to provide input and output between the sensor device 112 and a separate device (e.g., the processing device 130), and thus may be an example of an input/output (I/O) device in accordance with the present disclosure. However, in some examples the plurality of external pads 220 may be connected to a separate I/O device (see FIG. 3), such as a communication port, which in turn in is connected to the separate device. In either case, the I/O device is configured to output signal data from the plurality of electrically conductive pads 210 to the separate device and to receive response data from the separate device. In examples, the signal data may include data corresponding to the electrical characteristic (e.g., a value of the electrical characteristic, a change in value of the electrical characteristic over time, a value derived from the electrical characteristic, etc.). As will be described in more detail below with regard to FIGS. 4-5D, the separate device may operate or communicate with an ML algorithm (e.g., the ML model 140) which analyzes the signal data and generates the response data, the response data being indicative of a tissue type proximate respective ones of the plurality of electrically conductive pads 210.

In the examples of FIGS. 2B and 2C, a distal opening 240 can be seen. In the example of FIG. 2C, a cavity 250 can be seen. The distal opening 240 may be near the distal end 220 of the sensor body 200 as shown in FIGS. 2B and 2C, and/or may be at the distal end 220 of the device body as shown in FIG. 2C (but which would not be visible in the plan view of FIGS. 2A and 2B, even if present). In general, a plurality of the distal openings 240 may be present, including separate openings at the tip of the distal end 220 and at one or more locations along the length of the distal end 220. If the distal end 220 is generally cylindrical in cross-section and multiple distal openings 240 are present, the distal openings 240 may be located at the same or different positions in a circumferential direction and/or a longitudinal direction of the distal end 220. If the distal end 220 is polygonal in cross-section and multiple distal openings 240 are present, the distal openings 240 may be located on the same or different surfaces of the distal end 220. Moreover, distal openings 240 may take any shape, including longitudinal slots as shown in FIGS. 2B and 2C but also including other shapes such as circles, ovals, squares, rectangles, triangles, higher-order polygons, irregular shapes, and the like. Moreover, as shown in FIG. 2C, multiple distal openings 240 may be in communication with one another (e.g., a longitudinal slot opening in communication with an open end).

The distal openings 240 may be located at a set position, as illustrated in FIGS. 2B and 2C, or may be movable relative to the distal end 202. For example, the sensor body 200 may include a slidable or rotatable piece which may be moved to cover a portion of the illustrated slot, thereby to change an effective shape or dimension of the distal opening 240. In another example, the distal openings 240 may be a series of longitudinally-displaced holes (e.g., as in a flute) which may be selectively covered or uncovered by a slidable or rotatable piece.

The sensor body 200 may correspond in shape to 4 to 14 gauge (4-14 G) needle (e.g., an 8 G needle) with an outer diameter of about 4 mm or be dimensioned to be inserted within the 4-14 G needle, and may have a length of about 3-12 cm in an example. While not visible from the perspectives shown in FIGS. 2A-2C, the sensor body 200 may be equipped with a device for securing the sensor body 200 within the subject and/or a device for securing an instrument within a cavity of the sensor body 200. These securement devices may be a clamp, a hook, a pin, or any other device for locking the sensor body 200 and/or an instrument disposed therein at a particular position prior to performing any procedure. The sensor body 200 may also be configured with a gauge (e.g., a ruler or depth gauge) to convey the position of the sensor body 200 within the subject.

In implementations where the sensor device 112 is configured to be inserted into the insertable device 110 and the insertable device 110 includes the distal openings, at least some of the distal openings of the insertable device 110 may be positioned so as to expose the plurality of electrically conductive pads 210 to the subject tissue 120 when the sensor device 112 is inserted into the insertable device 110. Additionally or alternatively, in implementations where the instrument 114 is configured to be inserted into the sensor device 112 (whether the sensor device 112 is unitary with or separate from the insertable device 110), the distal openings of the sensor device 112 may be positioned so as to permit the instrument 114 to perform a procedure, for example by exposing the subject tissue 120 to the instrument 114.

FIG. 3 illustrates an example of the processing device 130. As illustrated, the processing device 130 includes a microprocessor 310, a memory 320, a feedback device 330, and an I/O device 340, which may be communicatively coupled to one another. The microprocessor 310, memory 320, feedback device 330, and I/O device 340 may reside within a common housing, as illustrated; alternatively, one or more of the microprocessor 310, memory 320, feedback device 330, and I/O device 340 may be distributed across several devices or housings in any combination. For example, in some implementations the feedback device 330 may instead reside in the vision system 150 (see FIG. 1).

The microprocessor 310 may include one or more electronic processors, each of which may include one or more processing cores, and/or one or more programmable hardware elements. The microprocessor 310 may be or include any type of electronic processing device, including but not limited to central processing units (CPUs), graphics processing units (GPUs), application specific integrated circuits (ASICs), field programmable gate arrays (FPGAs), microcontrollers, digital signal processors, or other devices capable of executing software instructions. In implementations where the processing device 130 has multiple microprocessors 310 and/or multiple processing cores, individual operations may be performed by any one or more of the microprocessors or processing cores, in series or parallel, in any combination.

The memory 320 may be any storage medium, including a non-volatile medium, e.g., a magnetic media or hard disk, optical storage, or flash memory; a volatile medium, such as system memory, e.g., random access memory (RAM) such as dynamic RAM (DRAM), synchronous dynamic RAM (SDRAM), static RAM (SRAM), extended data out (EDO) DRAM, extreme data rate dynamic (XDR) RAM, double data rate (DDR) SDRAM, etc.; or an installation medium, such as software media, e.g., a CD-ROM, or floppy disks, on which programs may be stored and/or data communications may be buffered. The term “memory” may also include other types of memory or combinations thereof. For the avoidance of doubt, cloud storage is contemplated in the definition of memory.

The memory 320 may be a non-transitory computer-readable medium which stores instructions that are executable by the microprocessor 310. Execution of the instructions by the microprocessor 310 may be configured to cause the processing device 130 or another device operating under control of the processing device 130 (e.g., the insertable device 110) to perform one or more operations, including but not limited to processing operations set forth herein (e.g., the operations of FIGS. 4 and/or 7, described in more detail below). For example, execution of the instructions may cause the system 100 to perform operations including receiving an electrical characteristic (e.g., a value measured by the insertable device 110), applying a k-nearest-neighbor (kNN) classification algorithm to the electrical characteristic, determining a tissue type based on a classification output from the kNN classification algorithm, and outputting an indication of the tissue type. In another example, execution of the instructions may cause the system 100 to perform operations including sensing an electrical characteristic, outputting signal data to a ML model (e.g., the ML model 140), and receiving response data generated by the ML model.

The feedback device 330 is configured to generate a feedback and provide the feedback to an operator of the system 100, either alone or in conjunction with the vision system 150. The feedback may be or include a haptic feedback, a visual feedback, an auditory feedback, or combinations thereof. The feedback is configured to indicate whether a portion of the subject tissue 120 (e.g., a portion of the tissue adjacent a portion of the insertable device 110, such as adjacent one or more of the electrically conductive pads 210) corresponds to a particular tissue type. In this manner, the feedback is configured to inform the operator that the insertable device 110 is or is not properly positioned with respect to a target tissue (e.g., a target nerve). The feedback device 330 may include a processing element, and in some implementations may be included within the microprocessor 310.

The I/O device 340 is configured to provide communication between the processing device 130 and other components of the system 100. For example, the I/O device 340 may be in communication with the plurality of external pads 220 (see FIGS. 2A-2C) to receive measurement data from the insertable device 110 and/or to provide response data to the insertable device 110, may be in communication with the vision system 150, for example to provide feedback to an operator of the system 100; may be in communication with one or more additional computing devices such as cloud servers, a device operating the ML model 140, and so on. The I/O device 340 may include wired and/or wireless communication circuitry, I/O ports such as Universal Serial Bus (USB) ports, I/O drivers, and the like.

In some implementations, the I/O device 340 may include output devices configured to provide the feedback generated by the feedback device 130 to the user. These output devices may include, but are not limited to, an audio device (e.g., a speaker) to output an audible signal, an illumination device (e.g., a light emitting diode (LED), a display such as a liquid crystal display (LCD) or organic light-emitting display (OLED), etc.) to output a visual signal, a haptic feedback device (e.g., an eccentric rotating mass (ERM), a piezoelectric actuator, etc.) to output a haptic signal, and the like. In other implementations, the vision system 150 may additionally or alternatively include the output devices and thus provide the feedback generated by the feedback device 130 to the operator. Either the I/O device 340, the vision system 150, or both may include a graphical user interface (GUI) to provide visual feedback to the operator.

The ML model 140 is configured to receive data from the sensor 112 of the insertable device 110, either directly or indirectly via the processing device 130, and to perform various operations on the data to determine characteristics of the subject tissue 120. FIG. 4 illustrates an example of operations performed by the ML model 140. The operations of FIG. 4 may be performed under the control of the processing device 130, or may be performed by the processing device 130 if the ML model 140 is included within the processing device 130. As noted above, where the processing device 130 (or any other device which houses the ML model 140) includes multiple processors or processing cores, individual ones of the operations or sub-operations of FIG. 4 may be performed by the same or different processors or processing cores in any combination.

At operation 410, the ML model 140 receives a measurement value of an electrical characteristic from an insertable device that has been inserted in a subject tissue (e.g., the insertable device 110 inserted in the subject tissue 120). The measurement value may be received in the form of a raw signal from measurement components (e.g., the plurality of electrically conductive pads 210) of the insertable device, and/or may include formatted data generated by an intermediate device (e.g., the processing device 130) based on the raw measurement data. Thus, the measurement value may directly correspond to a value of the electrical characteristic itself (e.g., impedance as a function of frequency), or may be derived from the value itself. For example, the measurement value may be an average of several measurements, a fast Fourier transform (FFT) of the value, an area under the curve (AUC) of the value, a coherence analysis of the value, a regression analysis of the value, or combinations thereof. In some implementations, the measurement value is a two-dimensional data array corresponding to a value of an electrical characteristic as a function of a measurement parameter (e.g., impedance as a function of frequency).

At operation 420, the ML model 140 applies a classification algorithm to the electrical characteristic embodied by the measurement value. In one particular example, operation 420 includes applying a k-nearest-neighbor classification algorithm to the measurement value. The kNN classification algorithm may be configured to compare the measurement value to a set of pre-classified reference values, in which the pre-classified reference values correspond to known tissue types. The comparison may be in terms of distance (e.g., Euclidean distance) between the measurement value and a pre-classified reference value. The comparison of the measurement value and each reference value may be arranged in order of increasing distance, truncated to the k closest reference values. These closest reference values may be subjected to a majority-rule operation, such that the measurement value is classified as the tissue type of the majority of the k closest reference tissue types. However, the ML model 140 is not limited to a kNN classification algorithm; in other examples, the ML model 140 may be or include a support vector machine (SVM) model, an artificial neural network (ANN), a random decision forest (RDF) model, a naïve Bayes classifier, or combinations thereof.

Regardless of the particular type of ML model 140 used, at operation 430 the ML model 140 determines the tissue type and at operation 440 the ML model 140 outputs an indication of the determined tissue type. The indication may be in the form of feedback (e.g., one or more feedback type described above) and/or a signal which causes another device in the system 100 to generate the feedback.

The operations of FIG. 4 are not necessarily performed one after another in strict sequence. In some implementations, the operations of FIG. 4 may be performed continuously or continually, such that an iteration of operation 420 (for example) is performed in parallel with a subsequent iteration of operation 410. In another example, operation 410 may be repeated (or performed simultaneously) for a plurality of electrical characteristics, such as an impedance measurement for multiple pairs of the plurality of electrically conductive pads 210), after which operations 420-440 may be performed. In such implementations, the system 100 may be configured for real-time visualization and/or identification of the subject tissue 120.

Returning to FIG. 1, the vision system 150 may be any device or system capable of visualizing information for a user. For example, the vision system 150 may be a display associated with or connected to the processing device 130. The display may be an LCD, an LED-LCD, an OLED, a plasma display panel (PDP), a virtual reality (VR) display, an augmented reality (AR) display, an extended reality (XR) display, a GUI on any one or more of the listed display architectures, an array of indicators (e.g., an array of LEDs, an array of light bulbs, a segmented liquid crystal (LC) indicator), or combinations thereof. In one particular example, the vision system 150 may be a laparoscope. The vision system 150 may be configured to receive feedback (e.g., via the feedback device 330) and present the feedback to the user.

FIG. 5A illustrates one example of the system 100 in use; that is, with the insertable device 110 inserted into a subject. FIG. 5B illustrates an example of measurement data generated by the system 100 and processed by the ML model 140. FIGS. 5C-5D illustrate examples of feedback provided to an operator of the system 100. In FIG. 5A, the sensor device 112 is illustrated as corresponding to the example of FIG. 2A; however, this is solely for purposes of illustration and the situation shown in FIG. 3 is equally applicable to any other configuration of the sensor device 112 in accordance with the present disclosure. In FIG. 5A, the sensor body 200 has been inserted beneath the skin of the subject tissue 120, such that the plurality of electrically conductive pads 210 (of which the bottom ten are shown) are in a subcutaneous state. A nerve 500 is present in the subject tissue 120 and crosses the sensor body 200. As noted above, each electrically conductive pad 210 may be identified by an index number. In FIG. 5A, pads 12-16 are illustrated on the left and pads 28-32 are illustrated on the right. In the illustrated example, the nerve 500 is in contact with pads 15 and 30 and no other pads.

The electrically conductive pads 210 are configured to detect a value of the electrical characteristic in the vicinity thereof, or to detect a difference in the value of the electrical characteristic between two, three, or four electrically conductive pads 210. In the example of FIG. 5B, the output is a difference value of which the output from pairs of the electrically-conductive pads 210 are illustrated. In particular, FIG. 5B illustrates several measurements between pad 15 and pad 30, and between pad 30 and pad 31. The ML model 140 may be configured to receive a composite measurement data 510, which in the illustrated example is a measurement of impedance vs. frequency visualized on a linear-linear scale. The horizontal axis of the composite measurement data 510 runs from 0 kHz to 50 kHz, and the vertical axis of the composite measurement data runs from −40 kΩ to 5 kΩ. The ML model 140 may be configured to classify the composite measurement data 510 into different groups, of which a first data group 512 and a second data group 514 are shown, both visualized on a linear-linear scale. The horizontal axis of the first data group 512 runs from 0 kHz to 50 kHz, and the vertical axis of the first data group 512 runs from 12 KΩ to 2 kΩ. The horizontal axis of the second data group 514 runs from 0 kHz to 50 kHz, and the vertical axis of the second data group 514 runs from −40 kΩ to −5 kΩ. Based on the characteristics of the respective data within each group, the ML model 140 may then be configured to determine which tissue types are present in the area near or between those pads 210 between which the measurements were taken. In this example, the ML model 140 may be configured to determine that the curve of the first data group 512 corresponds to pad 30 being proximate to nerve tissue and pad 31 being proximate to fat tissue (a “nerve-fat” classification); and to determine that the curve of the second data group 514 corresponds to the pad 30 being proximate to nerve tissue and pad 15 being proximate to nerve tissue (a “nerve-nerve” classification). Based on these determinations in combination with determinations based on measurements from other pairs of the electrically conductive pads 210 (e.g., from all neighboring pairs) and/or measurements from each electrically conductive pad 210 itself (e.g., relative to ground), the ML model 140 may be configured to generate a feedback itself or in combination with the feedback device 330 (see FIG. 3).

The feedback example of FIG. 5C includes an indicator array panel 520, which includes a plurality of indicators 525. The indicator array panel 520 may be a physical device or a virtual device (e.g., as a GUI element). One indicator 525 may be present for each of the plurality of electrically conductive pads 210, although only a subset of indicators 525 are explicitly shown in FIG. 5C for ease of explanation. In physical-device implementations, each of the indicators 525 may be directly connected to a corresponding electrically conductive pad 210. The plurality of indicators 525 may selectively indicate the tissue type proximate corresponding ones of the plurality of electrically conductive pads 210, for example based on information received from the ML model 140. The indicators 525 may be arranged in any manner; however, visualization and interpretation of the feedback from indicators 525 may be facilitated if the plurality of indicators 525 are arranged in an array that mimics the array in which the plurality of electrically conductive pads 210 are arranged. In this example, indicators 525 are illumination devices (e.g., LEDs, light bulbs, LC indicators) which are illuminated when the corresponding electrically conductive pad 210 is determined to be proximate a nerve tissue. Because, as can be seen in FIG. 5A, the nerve 500 crosses pads 15 and 30, corresponding indicators 15 and 30 are illuminated while the remaining indicators 525 are unilluminated.

The feedback example of FIG. 5D includes a tissue map 530 which represents the determined configuration of the subject tissue 120 in the area of the sensor device 112. Different tissue types may be represented by different patterns, as illustrated in FIG. 5D, or by different colors. While FIG. 5D illustrates three different tissue types (fat, nerve, and muscle), in practical implementations fewer or more tissue types may be differentiable. The tissue map 530 includes a series of overlays 535 which may correspond to the locations of electrically conductive pads 210. Thus, the number of overlays 535 may be the same as the number of electrically conductive pads 210, although only a subset of the overlays 535 are shown for ease of explanation. In other examples, the tissue map 530 may instead show a heat map for a single tissue type, thereby to convey a probability that each block within the tissue map 530 corresponds to the single tissue type. Moreover, in an implementation the GUI may be configured to permit an operator to switch between different views; for example to toggle between the tissue map 530 shown in FIG. 5D and individual heat maps corresponding to the different tissue types represented in the tissue map 530.

Map elements not directly within an overlay 535 (and thus corresponding to tissue not directly in contact with an electrically conductive pad 210) may be determined in one or more of several different manners. For example, as the insertable device 110 is inserted into the subject tissue 120, the system 100 may continuously sense and identify tissue. Thus, as the electrically conductive pads 210 move through the subject tissue 120, the system 130 (e.g., by continuously invoking the ML model 140) may generate data points corresponding to portions of the subject tissue 120 that are not in contact with any electrically conductive pad 210 when the sensor device 112 is in its final position. In some examples, the system 100 may infer data from between the electrically conductive pads 210, for example by measuring an impedance difference between adjacent pads 210 (see FIG. 5B). In still other examples, the ML model 140 itself may be configured to determine a probability that each inter-overlay map element belongs to a particular tissue type.

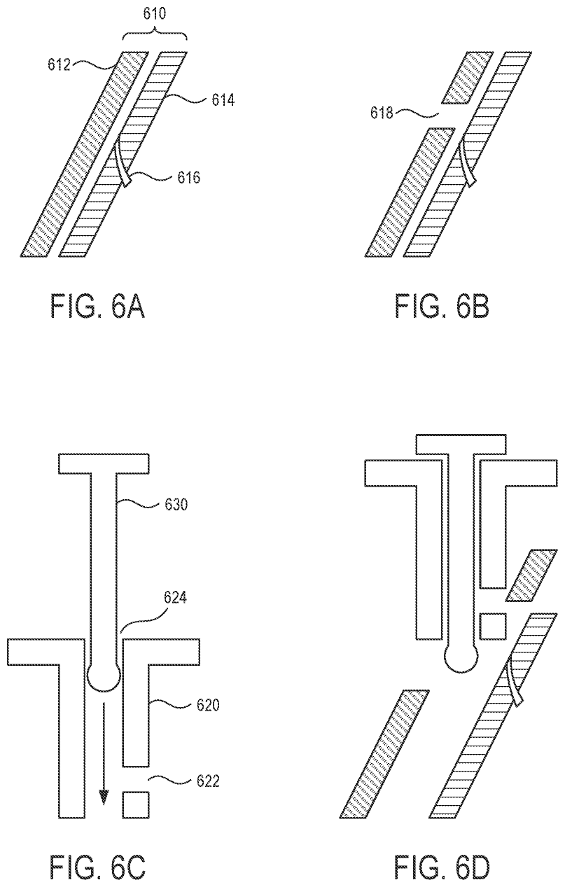

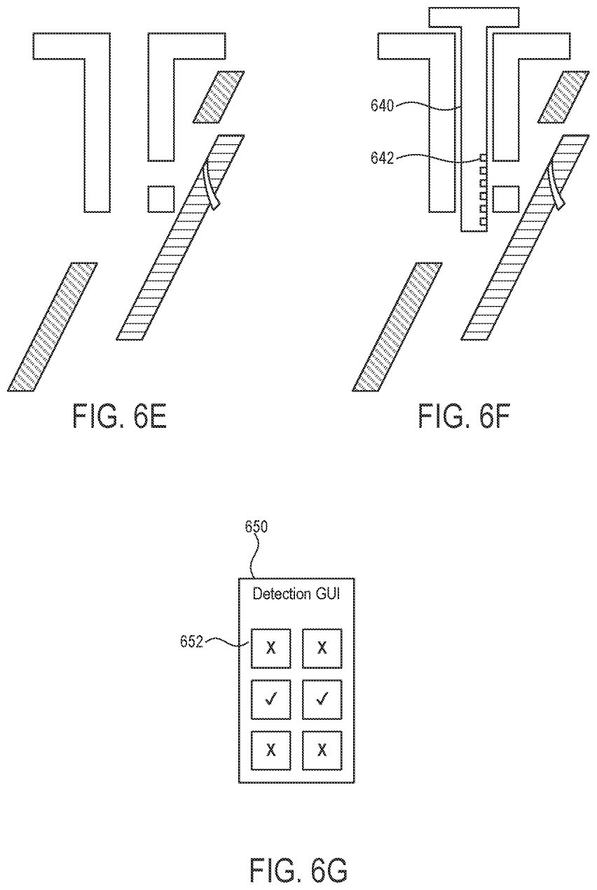

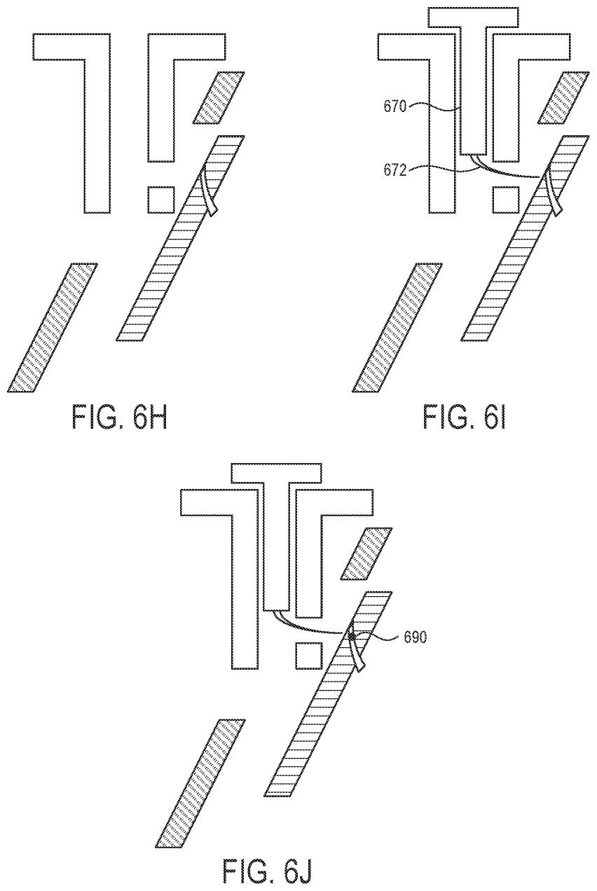

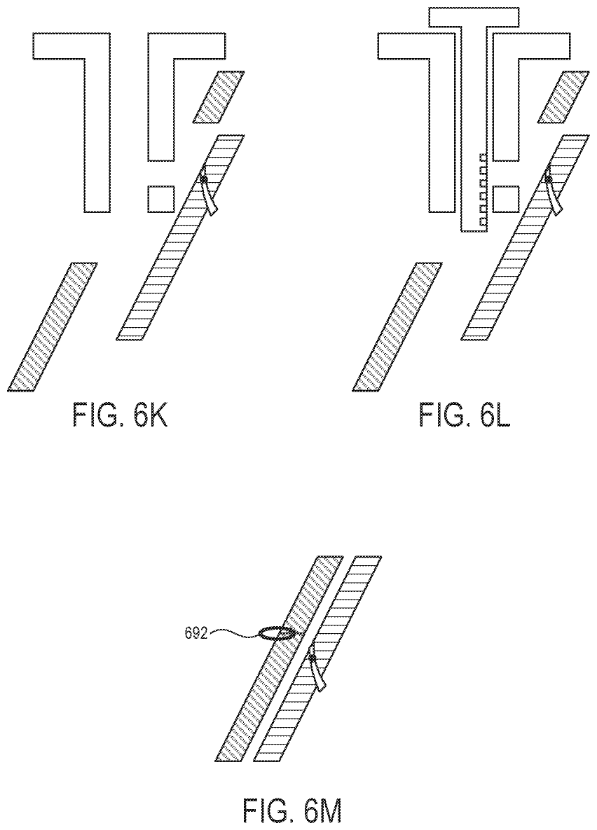

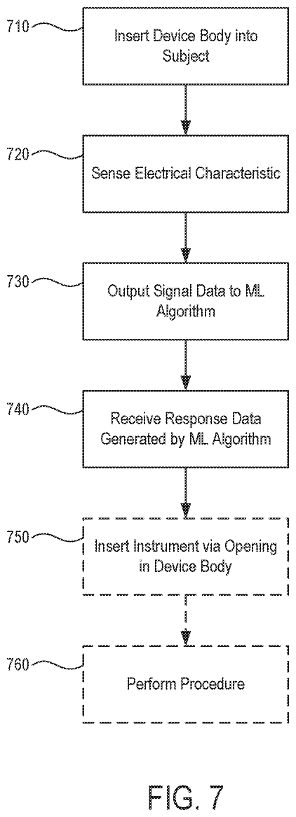

FIGS. 6A-6M and 7 illustrate a method of operating a medical device. In some examples, the medical device operated in the manner illustrated in FIGS. 6A-6M and 7 corresponds to the insertable medical device 110 illustrated in FIG. 1.

In FIG. 6A, a portion of tissue 610 is illustrated, which may correspond to the subject tissue 120 of FIG. 1. The tissue 610 includes dermal tissue 612, muscle tissue 614, and a nerve 616 extending through a portion of the muscle tissue 614. Other layers and tissue types may be present, and generally will depend on which portion of the body the tissue 610 is located. In one example, the tissue 610 may be a portion of a human head, in which case the other tissues may include subcutaneous tissue, galea aponeurotica, loose areolar tissue, temporoparietal fascia, superficial layer of the temporal fascia, interfacial fat pad, deep layer of the temporal fascia, temporal muscle, pericranium, and so on. In this example, the nerve 616 may be the frontotemporal branches of the facial nerve and may lie under the temporoparietal fascia. However, the present disclosure is not limited to human tissue and/or head tissue, and in other implementations may be animal tissue or synthetic tissue and may correspond to any body part.

An incision 618 is then made in the dermal tissue 612, as illustrated in FIG. 6B. The incision 618 may be made in the vicinity of the nerve 616, or may be made at a location that is relatively distant from the nerve 616 but reachable by the device which will be inserted therein (e.g., at a distance that is smaller than a length of the sensor device 112 and/or instrument 114 of FIG. 1). The method of operating the insertable medical device may be minimally invasive, and thus the incision 618 may be only large enough to permit the passage of the device therethrough.

With the incision 618 made (or, of course, prior to the incision 618 being made), the insertable device 620 may be prepared. The insertable device 620 is one example of the insertable device 110 illustrated in FIG. 1 and described above. As illustrated in FIG. 6C, the insertable device 620 has a device body which includes two distal openings 622 and a proximal opening 624, with a cavity extending through an interior of the device body of the insertable device 630. Of the two distal openings 622, a first distal opening 622 is located at the distal end of the insertable device 620 whereas a second distal opening 622 is located near the distal end of the insertable device 620 (e.g., in a sidewall of the insertable device 620). A first instrument 630 (one example of the instrument 114 of FIG. 1) having a blunt dissection tip is inserted into the insertable device 620 via the proximal opening 624.

With both the tissue 610 and the insertable device 620 prepared, the method of operating the device illustrated in FIG. 7 may begin. At operation 710, the insertable device 620 is inserted subcutaneously into the subject. This is also illustrated in FIG. 6D, in which the blunt dissection tip of the first instrument 630 separates the dermal tissue 612 from the muscle tissue 614 and holds the respective tissue in place. With the insertable device 620 in place, the first instrument 630 is removed from the cavity via the proximal opening 624, as shown in FIG. 6E. Subsequently, as shown in FIG. 6F, a detection tool 640 which includes a plurality of electrically conductive pads 642 is inserted into the insertable device 620 via the proximal opening 624, such that at least some of the plurality of electrically conductive pads 642 are exposed to the muscle tissue 614 via the second distal opening 622. The detection tool 640 is one example of the sensor device 112 illustrated in FIGS. 1 and 2A-2C. As noted above, in some implementations the detection tool 640 may be integral with the insertable device 620, in which case the plurality of electrically conductive pads 642 may be formed directly on the device body and the operation illustrated in FIG. 6E may be omitted. In either case, each of the plurality of electrically conductive pads 642 are located at a known position relative to the insertable device 620 (e.g., a first predetermined distance from the proximal opening 624 and a second predetermined distance from one of the distal openings 622).

At operation 720, the plurality of electrically conductive pads 642 sense an electrical characteristic of the tissue. Each electrically conductive pad 642 may separately sense the electrical characteristic, for example to provide information regarding whether a particular electrically conductive pad 642 is in contact with a particular tissue type. Alternatively, pairs of electrically conductive pads 642 may be used to sense a change in the electrical characteristic from pad to pad. Sensing may be repeated multiple times (e.g., five) per electrically conductive pad 642 or pair, and each sensing may be performed at a range of frequencies. Regardless, at operation 730 the detection tool 640 next outputs signal data from the plurality of electrically conductive pads 642 to a device operating an ML algorithm, which may be the same as or similar to the processing device 130 and/or the ML model 140 illustrated in FIG. 1. The signal data may include data corresponding to the measured electrical characteristic.

The ML algorithm may analyze the signal data to determine what tissue type is present proximate each of the plurality of electrically conductive pads 642. At operation 740, the ML algorithm may generate a response data indicative of the tissue type, which will be received by the insertable device 620 or by another device (e.g., a vision system such as the vision system 150 illustrated in FIG. 1). The response data may be interpreted and presented to an operator of the insertable device 620 using a detection GUI 650, as illustrated in FIG. 6G. The detection GUI 650 may be the same as or similar to the indicator array panel 520 of FIG. 5C or the tissue map 530 of FIG. 5D. As illustrated in FIG. 6G, the detection GUI 650 includes a plurality of indicators 652 (e.g., as in FIG. 5B) which respectively indicate whether the target tissue type is proximate corresponding electrically conductive pads 642. The operator may manipulate the detection tool 640 at this point to seek the target tissue type, consulting the detection GUI 650 to determine when the detection tool 640 is properly positioned.

If the detection tool 640 is separate from the insertable device 620, it may then be removed as shown in FIG. 6H. However, because the insertable device 620 has been secured, the insertable device 620 will not move relative to the target tissue (e.g., the nerve 616). With the insertable device 620 in place, a procedure (e.g., a surgical procedure) may be performed. In an example, at operation 750 a second instrument 670 may be inserted into the insertable device 620 via the proximal opening 624, as illustrated in FIG. 6I. The second instrument 670 may be at least one of a cautery tool, an injection needle, an incisor tool, a blade, a scissor tool, a hook tool, an ablation tool, a Doppler probe, a cryo probe, a sharp device body tip, or a blunt device body tip. In the illustrated example, the second instrument 670 is a cautery tool with a cautery tip 672 at a distal end thereof, such that the cautery tip 672 may be passed through the second distal opening 622 of the insertable tool 620.

The second instrument 670 is inserted to a predetermined depth, in which the predetermined depth is such that the cautery tip 672 is at a location of the nerve 616 as previously identified by the detection tool 640. The depth of insertion may be determined using cameras and/or a depth gauge present on the insertable device 620 and/or the second instrument 670 itself.

At this point, operation 760 of performing the surgical procedure itself may occur. As illustrated in FIG. 6J, the cautery tip 672 may cauterize a portion of the nerve 616, thereby creating a transection 690. The second instrument 670 may be withdrawn from the insertable device 620 as illustrated in FIG. 6K. The operator may then use the detection tool 640 once more to check that the nerve was properly transected by (if the detection tool 640 is separate from the insertable device 620) reinserting the detection tool 640 via the proximal opening of the insertable device 620. The operator may confirm that the detection tool 640 is properly positioned (e.g., using the cameras or depth gauge) and consult the detection GUI 650 to confirm that the transection 690 is properly and fully formed, as illustrated in FIG. 6L.

With the surgical procedure completed, the operator may remove the insertable device 620 and close the incision 618, for example with a suture 692 as shown in FIG. 6M.

In the above examples of the present disclosure in which the plurality of electrically conductive pads 210 and/or 642 are on a separate device from the insertable device 100 and/or 620, the sensor device 112 and/or the detection tool 640 are removed in order to insert the instrument 114 and/or 670. However, in other examples the sensor device may be configured to provide access to the instrument without a need to remove the sensor device.

The present invention may take any one or more of the following configurations:
1. An insertable medical device, comprising: a device body extending from a proximal end to a distal end and configured to be insertable into a subject; a proximal opening at the proximal end of the device body; a distal opening at or near the distal end of the device body; a cavity extending through the device body and in communication with the proximal opening and the distal opening, the cavity and the proximal opening being dimensioned to receive an instrument therein; a plurality of electrically conductive pads configured to be positioned a first predetermined distance from the proximal opening and a second predetermined distance from the distal opening, and configured to sense an electrical characteristic of internal tissue of the subject when the distal end is inserted into the subject; and an input/output (I/O) device configured to: output signal data from the plurality of electrically conductive pads to a processing device, the signal data including data corresponding to the electrical characteristic of the subject, and receive response data differentiating a tissue type proximate respective ones of the plurality of electrically conductive pads based on an analysis of the signal data.
2. The device of configuration 1, wherein the response data includes predictive data generated by a machine learning (ML) algorithm operated by the processing device.
3. The device of configuration 1 or 2, wherein the response data differentiates a nerve tissue type from a non-nerve tissue type.
4. The device of any one of configurations 1 to 3, further comprising a detection tool including the plurality of electrically conductive pads, wherein the detection tool is dimensioned to be received in the cavity via the proximal opening.
5. The device of any one of configurations 1 to 3, wherein the plurality of electrically conductive pads are formed on a surface of the device body.
6. The device of any one of configurations 1 to 5, wherein the plurality of electrically conductive pads are arranged in an array.
7. The device of any one of configurations 1 to 6, further comprising a securing element configured to secure an instrument, that is received in the cavity via the proximal opening, at a predetermined position within the cavity.
8. The device of any one of configurations 1 to 7, wherein the distal opening is one of a plurality of distal openings formed at or near the distal end of the device body.
9. The device of any one of configurations 1 to 8, wherein a position of the distal opening is movable relative to the distal end of the device body.
10. The device of any one of configurations 1 to 9, wherein the I/O device is further configured to output a feedback signal to an operator of the medical device based on the response data.
11. The device of any one of configurations 1 to 10, wherein the feedback signal is at least one of a haptic feedback signal, an auditory feedback signal, or a visual feedback signal.
12. The device of any one of configurations 1 to 11, wherein the electrical characteristic is a local impedance.
13. The device of any one of configurations 1 to 12, wherein the device body is minimally invasive.
14. The device of any one of configurations 1 to 13, wherein the insertable medical device is a nerve detection probe.
15. The device of any one of configurations 1 to 13, wherein the insertable medical device is configured to receive a nerve detection probe via the proximal opening, wherein the nerve detection probe includes the plurality of electrically conductive pads.
16. A method of operating a medical device, the method comprising: inserting a device body into a subject, wherein the device body includes a cavity extending from a proximal opening of the device body to a distal opening of the device body; sensing, by a plurality of electrically conductive pads located a first predetermined distance from the proximal opening and located a second predetermined distance from the distal opening, an electrical characteristic of the subject; outputting signal data from the plurality of electrically conductive pads to a processing device, the signal data corresponding to the electrical characteristic of the subject; and receiving response data differentiating a tissue type proximate respective ones of the plurality of electrically conductive pads based on analysis of the signal data.
17. The method of configuration 16, further comprising analyzing the signal data with a machine learning (ML) algorithm, and generating the response data by the ML algorithm.
18. The method of configuration 16 or 17, further comprising: inserting a detection tool into the cavity via the proximal opening, wherein the detection tool includes the plurality of electrically conductive pads.
19. The method of any one of configurations 16 to 18, further comprising: inserting an instrument into the cavity via the proximal opening; and securing the instrument at a predetermined position within the cavity.
20. The method of configuration 19, further comprising: performing a surgical procedure using the instrument.
21. The method of configuration 19 or 20, wherein the instrument is at least one of a cautery tool, an injection needle, an incisor tool, a blade, a scissor tool, a hook tool, an ablation tool, a Doppler probe, a cryo probe, a sharp device body tip, or a blunt device body tip.
22. The method of any one of configurations 16 to 21, further comprising: providing a feedback to an operator of the medical device based on the response data, wherein the feedback is configured to indicate a location of the medical device relative to a target tissue type.
23. The method of any one of configurations 16 to 22, further comprising: providing a feedback to an operator of the medical device based on the response data, wherein the feedback is configured to indicate a location of the medical device relative to a previously-stored reference location.
24. The method of any one of configurations 16 to 23, further comprising: selectively moving the plurality of electrically conductive pads toward or away from a position in which the plurality of electrically conductive pads are located the second predetermined distance from the distal opening.
25. The method of any one of configurations 16 to 24, further comprising: applying, by at least one of the plurality of electrically conductive pads, a voltage and/or a current to the subject.
26. A method of differentiating tissue, the method comprising: receiving signal data from a plurality of electrically conductive pads located a first predetermined distance from a proximal opening in a device body and located a second predetermined distance from a distal opening in the device body, the plurality of electrically conductive pads being located under a surface of a skin of a subject; analyzing the signal data to distinguish electrical characteristics of different structures internal to the subject; and generating an output that indicates a target tissue is proximate respective ones of the plurality of electrically conductive pads based on analysis of the signal data.
27. The method of configuration 26, wherein the target tissue is a nerve.
28. The method of configuration 26 or 27, wherein the device body includes a cavity configured to receive a detection probe inserted into the cavity via the proximal opening, wherein the detection probe includes the plurality of electrically conductive pads.
29. The method of any one of configurations 26 to 28, wherein the device body includes a cavity configured to receive an instrument into the cavity via the proximal opening and the output is configured to guide the instrument positioning via the cavity.
30. The method of configuration 29, wherein the instrument is a surgical tool.
31. The method of configuration 29 or 30, wherein the instrument is at least one of a cautery tool, an injection needle, an incisor tool, a blade, a scissor tool, a hook tool, an ablation tool, a Doppler probe, a cryo probe, a sharp device body tip, or a blunt device body tip.
32. The method of any one of configurations 26 to 31, wherein the output includes a feedback configured to indicate a location of the medical device relative to the target tissue.
33. The method of any one of configurations 26 to 32, wherein the output includes a feedback configured to indicate a location of the medical device relative to a previously-stored reference location.
34. The method of any one of configurations 26 to 33, further comprising analyzing the signal data as the plurality of electrically conductive pads are moved toward or away from a position in which the plurality of electrically conductive pads are located the second predetermined distance from the distal opening.
35. The method of any one of configurations 26 to 34, wherein the signal data is responsive to applying, by at least one of the plurality of electrically conductive pads, a voltage and/or a current to the subject.
36. A method of identifying a tissue, comprising: receiving, from an insertable medical device inserted in a subject tissue, a measurement value of an electrical characteristic; applying a machine learning (ML) model to the measurement value; determining, by the ML model, a tissue type based on the measurement value; and outputting an indication of the tissue type.
37. The method of configuration 36, wherein the ML model is configured to apply a k-nearest-neighbor (kNN) algorithm to the measurement value and to output a classification of the subject tissue.
38. The method of configuration 36 or 37, wherein outputting the indication of the tissue type includes displaying a heatmap indicating a probability of a target type in a vicinity of a portion of the insertable medical device.
39. The method of any one of configurations 36 to 38, wherein outputting the indication of the tissue type includes causing a haptic feedback to an operator of the insertable medical device, the haptic feedback indicating whether a portion of the subject tissue corresponds to a target tissue type.
40. The method of any one of configurations 36 to 39, wherein outputting the indication of the tissue type includes outputting an audible signal, the audible signal indicating whether a portion of the subject tissue corresponds to a target tissue type.
41. The method of any one of configurations 36 to 40, wherein outputting the indication of the tissue type includes selectively illuminating an illumination device based on a determination whether a portion of the subject tissue corresponds to a target tissue type.
42. The method of any one of configurations 36 to 41, wherein the electrical characteristic is an impedance.
43. The method of any one of configurations 36 to 42, wherein the electrical characteristic is an impedance difference between two, three, or four portions of the subject tissue.

Other examples and uses of the disclosed technology will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its embodiments.

What is claimed is:

1. An insertable medical device, comprising:
a device body extending from a proximal end to a distal end and configured to be insertable into a subject;
a proximal opening at the proximal end of the device body;
a distal opening at or near the distal end of the device body;
a cavity extending through the device body and in communication with the proximal opening and the distal opening, the cavity and the proximal opening being dimensioned to receive an instrument therein;
a plurality of electrically conductive pads configured to be positioned a first predetermined distance from the proximal opening and a second predetermined distance from the distal opening, and configured to sense an electrical characteristic of internal tissue of the subject when the distal end is inserted into the subject; and
an input/output (I/O) device configured to:
output signal data from the plurality of electrically conductive pads to a processing device, the signal data including data corresponding to the electrical characteristic of the subject, and
receive response data differentiating a tissue type proximate respective ones of the plurality of electrically conductive pads based on an analysis of the signal data.

2. The probe of claim 1, wherein the response data includes predictive data generated by a machine learning (ML) algorithm operated by the processing device.

3. The probe of claim 1, wherein the response data differentiates a nerve tissue type from a non-nerve tissue type.

4. The device of claim 1, further comprising a detection tool including the plurality of electrically conductive pads, wherein the detection tool is dimensioned to be received in the cavity via the proximal opening.

5. The device of claim 1, wherein the plurality of electrically conductive pads are formed on a surface of the device body.

6. The device of claim 1, wherein the plurality of electrically conductive pads are arranged in an array.

7. The device of claim 1, further comprising a securing element configured to secure an instrument, that is received in the cavity via the proximal opening, at a predetermined position within the cavity.

8. The device of claim 1, wherein the distal opening is one of a plurality of distal openings formed at or near the distal end of the device body.

9. The device of claim 1, wherein a position of the distal opening is movable relative to the distal end of the device body.

10. The device of claim 1, wherein the I/O device is further configured to output a feedback signal to an operator of the medical device based on the response data.

11. The device of claim 1, wherein the feedback signal is at least one of a haptic feedback signal, an auditory feedback signal, or a visual feedback signal.

12. The device of claim 1, wherein the electrical characteristic is a local impedance.

13. The device of claim 1, wherein the device body is minimally invasive.

14. The device of claim 1, wherein the insertable medical device is a nerve detection probe.

15. The device of claim 1, wherein the insertable medical device is configured to receive a nerve detection probe via the proximal opening, wherein the nerve detection probe includes the plurality of electrically conductive pads.

16. A method of differentiating tissue, the method comprising:
receiving signal data from a plurality of electrically conductive pads located a first predetermined distance from a proximal opening in a device body and located a second predetermined distance from a distal opening in the device body, the plurality of electrically conductive pads being located under a surface of a skin of a subject;
analyzing the signal data to distinguish electrical characteristics of different structures internal to the subject; and
generating an output that indicates a target tissue is proximate respective ones of the plurality of electrically conductive pads based on analysis of the signal data.

17. The method of claim 16, wherein the target tissue is a nerve.

18. The method of claim 16, wherein the device body includes a cavity configured to receive a detection probe inserted into the cavity via the proximal opening, wherein the detection probe includes the plurality of electrically conductive pads.

19. The method of claim 16, wherein the device body includes a cavity configured to receive an instrument into the cavity via the proximal opening and the output is configured to guide the instrument positioning via the cavity.

20. The method of claim 19, wherein the instrument is a surgical tool.

21. The method of claim 19, wherein the instrument is at least one of a cautery tool, an injection needle, an incisor tool, a blade, a scissor tool, a hook tool, an ablation tool, a Doppler probe, a cryo probe, a sharp device body tip, or a blunt device body tip.

22. The method of claim 16, wherein the output includes a feedback configured to indicate a location of the medical device relative to the target tissue.

23. The method of claim 16, wherein the output includes a feedback configured to indicate a location of the medical device relative to a previously-stored reference location.

24. The method of claim 16, further comprising analyzing the signal data as the plurality of electrically conductive pads are moved toward or away from a position in which the plurality of electrically conductive pads are located the second predetermined distance from the distal opening.

25. The method of claim 16, wherein the signal data is responsive to applying, by at least one of the plurality of electrically conductive pads, a voltage and/or a current to the subject.

26. A method of identifying a tissue, comprising:
receiving, from an insertable medical device inserted in a subject tissue, a measurement value of an electrical characteristic;
applying a machine learning (ML) model to the measurement value;
determining, by the ML model, a tissue type based on the measurement value; and
outputting an indication of the tissue type.

27. The method of claim 26, wherein the ML model is configured to apply a k-nearest-neighbor (kNN) algorithm to the measurement value and to output a classification of the subject tissue.

28. The method of claim 26, wherein outputting the indication of the tissue type includes displaying a heatmap indicating a probability of a target type in a vicinity of a portion of the insertable medical device.

29. The method of claim 26, wherein outputting the indication of the tissue type includes causing a haptic feedback to an operator of the insertable medical device, the haptic feedback indicating whether a portion of the subject tissue corresponds to a target tissue type.

30. The method of claim 26, wherein outputting the indication of the tissue type includes outputting an audible signal, the audible signal indicating whether a portion of the subject tissue corresponds to a target tissue type.

31. The method of claim 26, wherein outputting the indication of the tissue type includes selectively illuminating an illumination device based on a determination whether a portion of the subject tissue corresponds to a target tissue type.

32. The method of claim 25, wherein the electrical characteristic is an impedance.

33. The method of claim 25, wherein the electrical characteristic is an impedance difference between two, three, or four portions of the subject tissue.
